(19) United States
(12) Patent Application Publication
(10) Pub. No.: US 2022/0295969 A1
(43) Pub. Date: Sep. 22, 2022

(54) COMPLETE PORTABLE ORAL CARE AND HYGIENE SYSTEM

(71) Applicant: Byron Patricio ROBAYO ARROYO, Quito (EC)

(72) Inventor: Byron Patricio ROBAYO ARROYO, Quito (EC)

(21) Appl. No.: 17/287,877

(22) PCT Filed: Dec. 7, 2018
(86) PCT No.: PCT/IB2018/059778

§ 371 Date: Apr. 22, 2021

(51) Int. Cl. A45D 44/18; A46B 17/04; A46B 9/04; A46B 11/00; A61C 15/04; A46B 5/00

(52) U.S. Cl. CPC A45D 44/18; A46B 17/04; A46B 9/04; A46B 11/001; A46B 11/0093; A61C 15/043; A46B 5/0095; A46B 2200/1066

(57) ABSTRACT

A complete system for oral care and hygiene is revealed, easy portability, hygienically safe and comfortable to use, which facilitates the availability of a toothbrush, toothpaste and dental floss, all included in a coupled body, and allows to be carried with security and hygiene in wallets, clothing, and others; optimizing access at any time to a complete system for the care and cleaning of teeth and gums.

### TECHNICAL FIELD OF THE INVENTION

This invention belongs to field of oral care and hygiene, and refers to a system that comprises a portable toothbrush that is made up of three functional bodies, each of which has useful and complementary elements for comprehensive oral care.

### BACKGROUND OF THE INVENTION

According to the survey published by Delta Dental Oral (The largest health and quality of life Portal. ES. AAZSANTE.FR. https://es.aazsante.fr/research-reveals-deficiencies-in-americans-dental-health-habits-7869), seven out of ten Americans brush their teeth only twice a day, once at bedtime and once after getting up in the morning, and only 17% of Americans brush their teeth after lunch. According to these statistics published by Delta Dental Oral, only four out of ten Americans floss at least once a day and 20% never floss.

On the other hand, in the document “National Library of Medicine of the USA. Medline Plus. Adult dental care. Revised on May 2, 2018”, the diseases caused by inadequate hygiene of the mouth are described as follows:

“Tooth decay and gum disease are caused by plaque, a sticky combination of facteria and food. Plaque begins to build up on your teeth within minutes of eating. If teeth are not washed after eating, the plaque will cause tooth decay and gum disease. If you do not remove the plaque, it becomes a hard deposit called tartar that is trapped at base of tooth. Plaque and tartar irritate and inflame the gums. Bacteria and toxins cause gums to:
Get inflamed
Swell
Have greater sensitivity

Plaque and tartar can lead to a variety of problems:
Dental caries are holes that damage tooth's structure.
Gingivitis are swollen, inflamed, and bleeding gums.
Periodontitis is destruction of ligaments and bone that supports the tooth, which often leads to tooth loss.
Bad breath (halitosis).
Abscessed, pain; inability to use teeth.
Others health problems external to the mouth, from preterm birth to heart disease.”

Based on the above, the American Dental Association recommends brushing your teeth three times a day, that is, once after each meal, making sure to brush all teeth, on the inside, outside and chewing surface, paying special attention to the area where the tooth and gum meet.

Likewise, spaces that remain between teeth not only accumulate food, but also plaque that must be removed, for which it is recommended the use of dental floss that must be used at least once before brushing teeth, every time it serves to remove plaque between teeth and over gums.

Indeed, taking proper care of teeth and gums can help prevent problems such as cavities, gingivitis and periodontitis, but unfortunately, and due, among other factors, fast rate of life, very few people brush their teeth after lunch, mainly as a result of lack of availability of a functional toothbrush that includes all elements required for adequate oral health and hygiene, and additionally to be easy to transport and use anywhere and anytime.

Respect to the above, different toothbrushes are known of the technical state of the art that are provided with fluids and systems for dispensing oral care agents (particularly toothpastes). For example, patent document US 2005/111905 discloses a combination of a toothbrush, toothpaste and a dental floss dispenser, where that product is designed to use a replacement cartridge and includes a transparent head cover with means to ventilate and dry the brush after use. Specifically, the disclosed product consists of an upper assembly (including transparent head cover, head/neck brush, suction plunger, plunger button, and feed tube), and lower assembly including toothpaste reservoir, bottom cover, base cap, thread reel and cutter. Now, pressing plunger button presses suction plunger creating a suction that draws paste out of toothpaste reservoir and deposits it in toothbrush head. Finally, floss included in the handle of toothpaste reservoir is extracted from the base cap and then removed using cutter.

On the other hand, document WO2011/109626 mentions an oral care implement that supplies toothpaste, which is characterized because it comprises a head that includes at least one dental cleaning element, a reservoir member for storing a fluid (toothpaste), at least one fluid outlet placed in the head, and channel fluidly coupled to storage member with the outlet, where channel transfer fluid at a first rate of fluid flow to the outlet and the outlet transfers fluid to a user.

For its part, document US2011/286784 discloses hand-held dental appliance that corresponds to a complete and portable dental care system. The appliance stores toothpaste supply container. The device has a removable cover to protect a toothbrush unit, toothpaste dispenser which is separately connected to container. The toothbrush unit has a brush head at the end of a shank that is attached to a cooperating container connector. When the brush unit and container connectors come together, a dispenser tip at container connector end fluidly communicates with a hole arranged within a matrix of bristles on toothbrush head, where such hole is overlapping by a set of elastic fins that allow toothpaste to flow towards bristles, but inhibits the reflux through the hole. For its part, protective cover includes a housing that receives and encloses toothbrush unit through an open end, while other end of the cover includes a hinged lid that covers dental floss dispenser contained within the cover when cover is closed. Thread lengths can be cut from thread dispenser by a cutter protruding from an opening adjacent to the lid.

Patent document US 2017/049220 discloses a dispensing toothbrush that is capable of storing and dispensing toothpaste and dental floss. The toothbrush includes a handle that has a chamber built to store toothpaste, dial, and floss dispenser that has floss axis, floss opening and floss cutter, neck, and head with bristles and head holes. For operation, dial is placed on the handle between thread dispenser and neck, so that by turning the dial, toothpaste is extruded through openings in the toothbrush head. According to inventors, revealed toothbrush can be provided in a compact size and is ideal for traveling, as it brings together multiple elements (toothbrush, toothpaste, and dental floss) in a single compact device.

All the aforementioned documents disclose toothbrushes including dispensers and fluid delivery systems (cream toothpaste), requiring elements such as fluid containing storage tanks and channels with capillaries means extending through one or more outlets in toothbrush head and allow to distribute fluid to bristles or filaments.

The problem that has been found in oral care devices in the mentioned documents consists in improper operation of capillary and storage system of toothpaste, given that toothpaste tends to dry on contact with air, which normally it produces at the moment of filling the storage container and at exit of the toothpaste, a fact that causes toothpaste to accumulate gradually which finally obstructs exit of the product, leaving the mechanism without operation.

Another problem observed lies in complexity of filling the storage device with toothpaste periodically as required, preserving the safety and hygiene conditions necessary for oral care.

The state of the art also shows the following devices that include toothbrushes where cameras have been added to the handle to provide toothpaste to improve tooth brushing process:
Patent document TW201735831 refers to a toothbrush with hollow handle that allows the storage of toothpaste in capsule form, where each capsule is housed in a separate cavity.
Patent document WO97/43924 refers to a toothbrush that consists of a bristle head and a handle that includes a removable support, inside which a dental floss spool and a toothpick are housed.
Patent document WO2015/171133 discloses an oral care system that includes a grip or handle and a dental head, where the handle comprises an internal cavity and a removable lid that is in special communication with the internal cavity of the handle. Such oral care system also includes a plurality of storage chambers in the handle, each of which contains a dose of an oral care material, and where each of the storage chambers can be individually activated to release the dose of oral care material in the removable cap.
Patent document KR101375548 refers to toothbrush that includes toothpaste in capsules, which are stored inside the handle of toothbrush to be used one by one by the user. Additionally, encapsulated toothpaste is characterized by having various scents and colors, which can induce tooth brushing in infants or children who are reluctant to brush their teeth.
Finally, patent document KR 2015/0060478 discloses an encapsulated toothpaste that is dispensed onto a portable toothbrush by inserting capsule into toothbrush head for later use.

So far, however, toothbrushes only refer to devices that include a head with bristles, coupled to a second body in the form of a handle for housing encapsulated toothpaste, but where such devices do not provide complete care systems, and oral hygiene that include all elements required for a correct oral cleaning, and that also guarantee an adequate disposition and dosage of toothpaste, as well as its safety and hygiene through a secure fit of storage container, also allowing adequate and effective portability of toothbrush.

### BRIEF DESCRIPTION OF THE INVENTION

Taking into account the problem that persisted in the prior state of the art, the applicant developed a complete system for oral care and hygiene that is easy to carry, hygienically safe and comfortable to use, which facilitates the availability of a toothbrush, toothpaste, and dental floss all included in coupled body, and also allows to be carried with security and hygiene in wallets, clothing, and others; optimizing access at any time to a complete system for the care and cleaning of teeth and gums.

In effect, the developed system is made up of three functional bodies,—upper, central and lower—, where each one of them has useful and complementary elements for comprehensive oral care, allowing in this way to efficiently solve the need to have a complete oral care and hygiene system that includes all elements required for correct oral cleaning, and that also guarantees an adequate provision and dosage of toothpaste.

### DESCRIPTION OF THE FIGURES

FIG. 1 shows the upper (A), central (B) and lower (C) bodies that make up complete oral care and hygiene system shown in the present invention.

FIG. 2 shows the upper body (A) of complete oral care and hygiene system shown here, as well as its constituent parts: Ventilation slots, adjustment clip, circular internal thread and draining element

FIG. 2.1 shows rear view of the upper body (A) showing ventilation slots and draining element.

FIG. 3 shows the central body (B) of complete oral care and hygiene system disclosed herein, and its constitutive parts: bristles or filaments, protruded curb, pill deposit element, upper coupling thread, and lower coupling thread.

FIG. 4 shows the lower body (C) of complete oral care and hygiene system disclosed here, and its constitutive parts: Storage container, coupling element with the central body, support stop pieces, extended container, impeller dental floss, hitch groove and pointed element.

### DETAILED DESCRIPTION OF THE INVENTION

The object of present invention is a complete oral care and hygiene system that is easy to carry, hygienically safe and comfortable to use, where such system facilitates the availability of an all inclusive toothbrush, toothpaste and dental floss, and allows carry it safely and hygienically in wallets, clothing and others, optimizing at any time access to a complete system for the care and cleaning of teeth and gums.

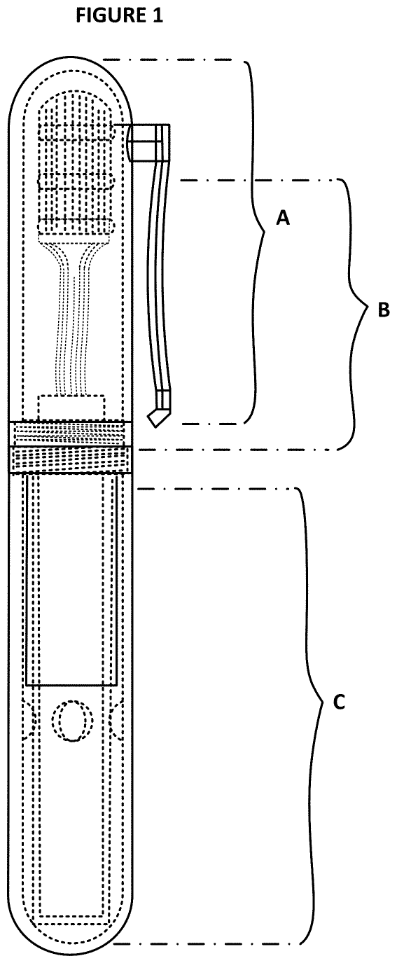

To achieve the object of the invention, complete portable oral hygiene and care system is made up of three bodies: upper, central and lower (FIG. 1).

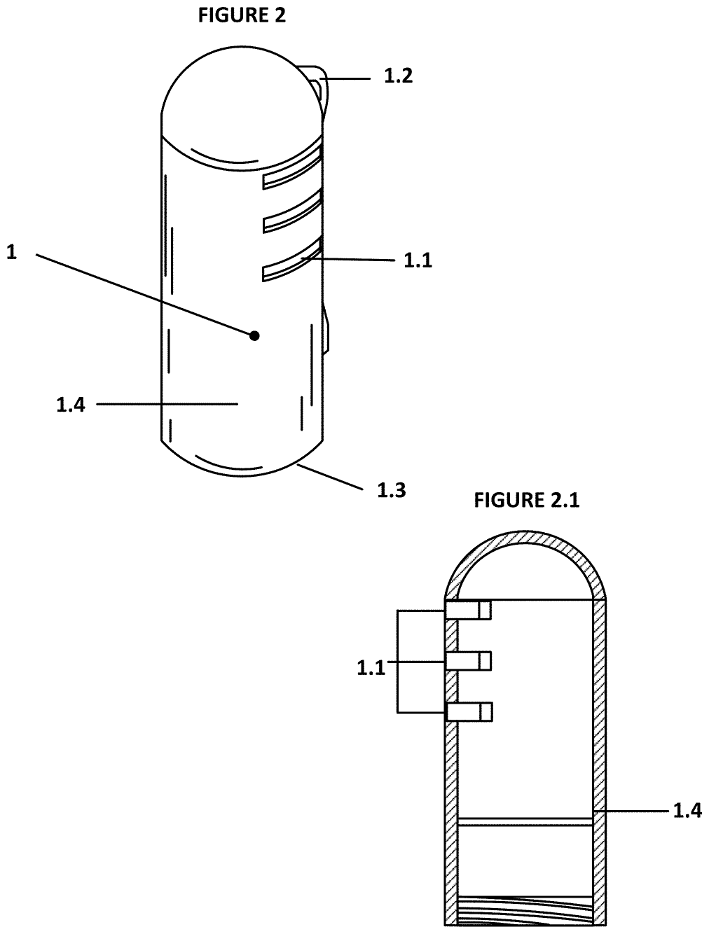

The upper body (FIGS. 1 and 2) is made up of a cap (1) that covers head of toothbrush and ensures that filaments or bristles are not in contact with the environment, thus preserving proper hygiene. The cap (1) also has grooves (1.1) that allow ventilation and drying of the head to eliminate humidity that occurs in filaments after brushing. The cap (1) additionally has a clip (1.2) that serves as a holding mechanism in garments, facilitating portability of oral care system of the invention. The cap (1) also has a coupling mechanism (1.3), preferably in shape like a circular internal thread, which allows toothbrush head to be hermetically closed to prevent the cap (1) from coming off and contaminating head of toothbrush and filaments.

The cap (1) has a draining implement (1.4) located inside the cap (1), above internal thread (1.3) and surrounding internal section of the cap (1), where such draining implement (1.4) allows squeeze filaments or bristles of toothbrush by friction that occurs between draining implement (1.4) and brush head's filaments at the entrance to the cap, facilitating to drain excess water.

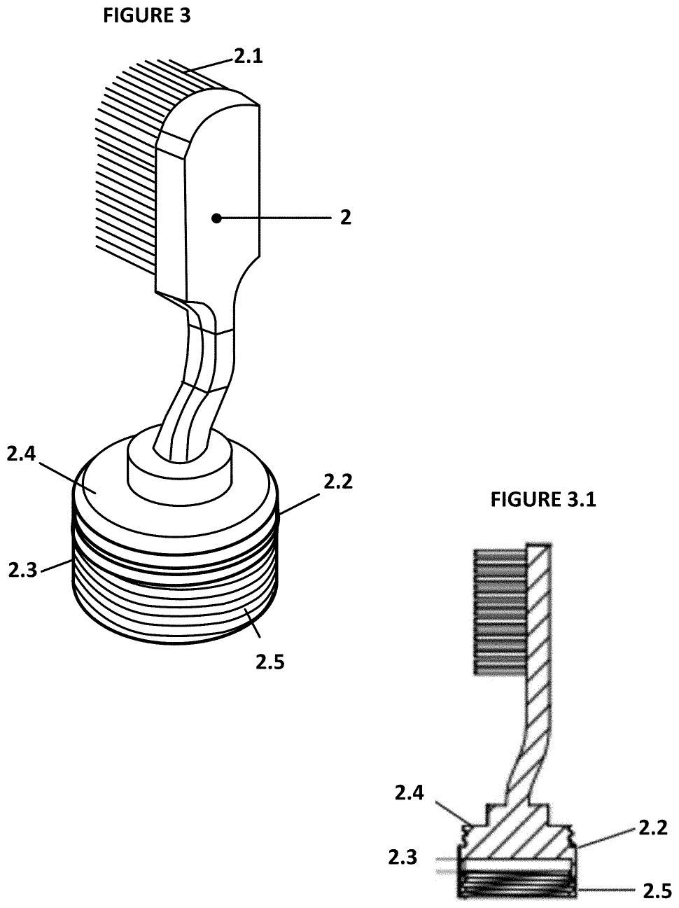

The central body (FIGS. 1 and 3) is made up of toothbrush head (2) that has bristles or filaments (2.1) that allow brushing teeth, and where filaments are arranged in the position and recommended quantity to ensure high brushing efficiency.

The brush head (2) additionally has a protruded curb (2.2) in its lower part that contains deposit element (2.3) whose diameter and volume correspond to the one allows the fitting of a single tooth tablet, so that when connecting the central body with receptacle (3) and subsequently slightly rotating the entire oral care system revealed here to a horizontal position, it is achieved that only one tooth tablet comes out at a time and it is placed exactly in the space provided for this effect in the reservoir (2.3), thus preventing abrupt exit of tablets and facilitating later distribution of solid toothpaste by units directly in the mouth.

The protruded curb (2.2) additionally serves as a bridge between upper and lower bodies and allows the entire system to be supported, facilitating the opening of each body independently. The central body also has a coupling (2.4) that allows adjusting the cap (1) with head of toothbrush (2). The deposit element (2.2) also has a coupling system that allows it to be adjusted to receptacle and allows the central body to join the lower body. The protruded curb has a lower coupling (2.5) with receptacle.

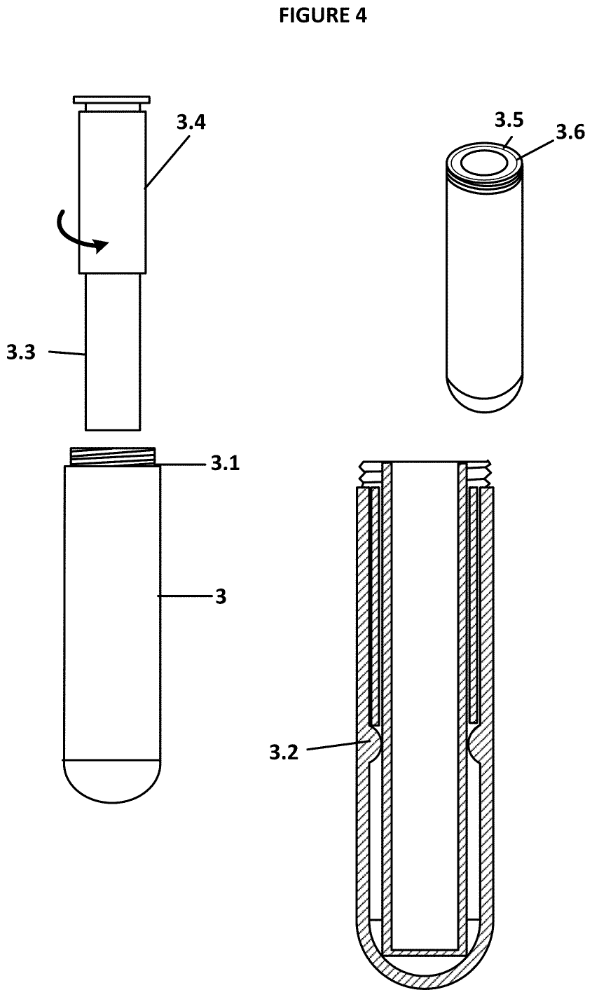

The lower body (FIGS. 1 and 4) is made up of a receptacle (3) that contains the following elements inside:
An extended container (3.3) that allows to store inside solid toothpastes, powders, tablets, gel pills, etc.
An impeller (3.4) that allows storing and providing dental floss, where such impeller (3.4) is attached above the extended container (3.3) as shown in FIG. 1.

Inside the receptacle (FIG. 1) there are also stop pieces (3.2) that support the impeller, in order to avoid dropping it into receptacle (3), but at the same time allowing a rotary movement to facilitate exit of dental floss through coupling groove (3.5). Stop pieces (3.2) also serve to stabilize extended container and allow it to remain fixed in the center of receptacle (3). The receptacle (3) comprises a coupling element that allows the lower body to be joined with the central body, also achieving closure of extended container which helps to keep tablets hermetically sealed, preserving their hygiene and safety.

The extended container (3.3) also has a coupling groove (3.5) in the upper part that allows dental floss to exit (3.6), with a pointed item that facilitates to cut off dental floss (3.6).

In a particular modality of the invention, the oral care system disclosed herein can be made entirely or partially with any type of polymer, plastic or resin, and include metal parts, biomaterials or biodegradable materials.

In another modality of the invention, clamping mechanisms for each of the bodies can be mechanically, by threads or with any other mechanism that ensures closure and also facilitates opening of each of the bodies separately.

In another modality, the draining implement (1.4) located above internal cap's thread (1) is at least a circle or semicircle that surrounds an internal section of the cap and whose diameter is greater than internal cap's thread.

In another modality, the cap (1) has a draining implement (1.4) located above internal thread, which is at least one protruded tip that surrounds an internal section of the cap (1) whose diameter is greater than the thread inside the cap (1).

In another modality of the invention, the system has replacements for toothbrush head, which can be exchanged once filaments have been worn by use, or by hygiene recommendations.

In another modality of the invention, it is suggested that once dental floss has run out there is a spare replacement for dental floss spool.

In another modality of the invention, the cap's clip can be removable or adjustable at different distances based on use requirements of the cap and toothbrush; and be located anywhere on the cap.

In one of the modalities, the cap may have, in addition to aeration slots, other mechanisms that allow ventilation of head and avoid moisture in filaments.

In one of the modalities, toothpaste can be made up of pills, tablets, powders, lozenges, pellets, or any other form of presentation that allows to be used for washing teeth. Likewise, solid toothpastes can contain active ingredients of botanical origin, as well as excipients that contribute with the cleaning and infiltration of active ingredients for a better teeth and gums cleaning.

From detailed description it can be seen that one of the advantages of the invention lies in the ease of the system of being able to be carrying in a hygienic and safe way, as a clip-on pen to carry, thanks to the cap clip that allows to secure the complete system in any type of garments. Another advantage lies in the use of solid toothpaste that eliminates problems of state of the art toothbrushes where the use of a cream or flowing toothpastes causes plugging or obstruction of storage receptacle and toothpaste's distribution capillaries towards toothbrush head.

The invention's system made with an independent extended container for housing solid toothpaste, where the opening that allows exit of solid toothpaste is located in the upper part of the container, has the advantage of facilitating outlet of pills (tablets, dragees, etc.) by units, solving the problem found in other devices that include solid toothpaste, where opening of the device is at lower end of the handle, causing pills to fall together due to gravity effects and produce contamination due to their manipulation.

It should be understood that although the invention has been described in conjunction with its specific modalities, the foregoing description and examples are illustrative nature and do not limit its scope. Other aspects, benefits and modifications will be apparent to those skilled in the art to which the invention belongs, and these aspects and modifications fall within the scope of invention in accordance with accompanying claims.

It is important to mention that regarding this date, the best method known to the applicant to put the aforementioned invention into practice is the one that is clear from the present invention description.

1-17. (canceled)

18. A dental care and oral hygiene system comprising:
an upper body comprising a toothbrush head;
a cap that covers the toothbrush head, the cap removable from the upper body and having a ventilation mechanism, a holding clip, a hermetic coupling mechanism, and a draining implement;
a central body having an attached toothbrush head that includes bristles or filaments, and having a lower portion that includes a protruded curb that contains a reservoir and includes a coupling for engaging the hermetic coupling mechanism of the cap; and
a lower body comprising
a receptacle including a support stop protruding from an inside wall of the receptacle,
a container received in the receptacle and configured to store solid toothpaste, and
a dental floss impeller coupled to the container.

19. The dental care and oral hygiene system of claim 18, wherein the draining implement surrounds an internal section of the cap and is located above the hermetic coupling mechanism.

20. The dental care and oral hygiene system according to claim 19, wherein the draining implement is located above the circular internal thread and comprises a protruded tip that surrounds an internal section of the cap, and wherein the internal section of the cap has a diameter greater than an inside diameter of the circular internal thread.

21. The dental care and oral hygiene system according to claim 19, wherein the draining implement is located above the circular internal thread, and wherein the draining implement extends circumferentially at least half way around an internal section of the cap, and wherein the internal section of the cap has a diameter greater than an inside diameter of the circular internal thread.

22. The dental care and oral hygiene system of claim 18, wherein the ventilation mechanism of the cap comprises slots located in an upper portion of the cap.

23. The dental care and oral hygiene system of claim 18, wherein the hermetic coupling mechanism comprises a circular internal thread.

24. The dental care and oral hygiene system of claim 18, wherein the protruding curb is coupled to the receptacle by an internal thread, and coupled to the cap by an external thread, the protruding curb serving as bridge between the upper and lower bodies.

25. The dental care and oral hygiene system of claim 18, wherein a diameter and a volume of the container allows a tablet of solid toothpaste to leave the container after connecting the central body with the receptacle and so that only one tablet can be placed in the container.

26. The dental care and oral hygiene system according to claim 18, wherein the receptacle has three support stops.

27. The dental care and oral hygiene system according to claim 18, wherein the dental floss impeller is configured to rotate about a central axis of the container, thereby allowing dental floss to unfold.

28. The dental care and oral hygiene system according to claim 1, wherein the container has on its surface a coupling groove and a pointed element.

29. The dental care and oral hygiene system according to claim 18, wherein each of the upper body, the cap, the central body, and the lower body is made with any type of polymer, plastic, or resin.

30. The dental care and oral hygiene system according to claim 18, wherein constituent elements include metal parts, biomaterials or biodegradable materials.

31. The dental care and oral hygiene system according to claim 18, wherein the toothbrush head is removably attached to the central body.

32. The dental care and oral hygiene system according to claim 18, wherein the holding clip of the cap is removable or adjustable at different distances from the cap according to needs of use.

33. The dental care and oral hygiene system according to claim 18, further comprising solid toothpaste housed in the container of the lower body, the solid toothpaste having a form selected from a pill, a tablet, a powder, a lozenge, or a pellet.

34. The dental care and oral hygiene system according to claim 33, wherein the solid toothpaste contains active ingredients of botanical origin, as well as excipients that contribute to cleaning and to infiltration of active ingredients into teeth and gums.
